(19) United States
(12) Patent Application Publication
(10) Pub. No.: US 2026/0294629 A1
(43) Pub. Date: Oct. 1, 2026

(54) SEGMENTED CALCIUM PHOSPHATE BONE SCAFFOLDS WITH SACRIFICIAL ZONES FOR MECHANO-TRANSDUCTIVE BONE REGENERATION AND METHODS OF MAKING AND USING THE SAME

(71) Applicant: Board of Regents, The University of Texas System, Austin, TX (US)

(72) Inventors: Teja Guda, San Antonio, TX (US); Anson Joo Leng Ong, San Antonio, TX (US); Gennifer Chiou, San Antonio, TX (US)

(21) Appl. No.: 19/634,001

(22) Filed: Mar. 30, 2026

Related U.S. Application Data
(60) Provisional application No. 63/780,414, filed on Mar. 30, 2025.

(51) Int. Cl. A61F 2/28; A61L 27/04; A61L 27/10; A61L 27/12; A61L 27/24; A61L 27/54; A61L 27/56; A61L 27/58

(52) U.S. Cl. CPC A61F 2/2846; A61L 27/04; A61L 27/10; A61L 27/12; A61L 27/24; A61L 27/54; A61L 27/56; A61L 27/58; A61F 2210/0004; A61F 2210/0061; A61F 2210/0076; A61L 2300/102; A61L 2300/112; A61L 2300/252; A61L 2300/412; A61L 2300/604

(57) ABSTRACT

This application describes scaffolds, particularly bone scaffolds, with localized changes in mechanical properties allowing for re-introduced stalled bone regeneration within critical sized defects. Utilizing mechanical stiffness and scaffold degradation initiated mechano-transductive pathways re-activates bone regeneration activity. Localized changes in mechanical properties in spatial and temporally controlled manner reinduces stalled bone regeneration.

### RELATED APPLICATIONS

This application claims priority to U.S. Provisional Patent Application 63/780,414 filed Mar. 30, 2025 which is incorporated herein by reference in its entirety.

### STATEMENT REGARDING FEDERALLY FUNDED RESEARCH

This invention was made with government support under R21-DE032179-01A1 awarded by the National Institutes of Health. The government has certain rights in the invention.

### FIELD

Embodiments of the present invention relate generally to the field of tissue engineering and regenerative medicine. More particularly, embodiments are directed to bone scaffolds and methods for their fabrication and use in the repair and regeneration of bone tissue, including critical-size bone defects in the craniofacial and craniomaxillofacial skeleton.

### BACKGROUND

Bone defects occur in craniofacial skeletons due to reasons such as traumatic injuries in the craniomaxillofacial skeleton,¹⁻³ bone loss due to disease or tooth extraction,^{4,5} congenital birth defects^{6,7} and the resection of bone tissue during cancer therapy.^{8,9} The treatment of these defects is often challenging because anatomical form is crucial to function in the craniofacial skeleton, both for successive implant placement and for the maintenance of facial aesthetics to avoid psychological stress in patients. Current use of autologous bone grafts, including transplantation of bony segments with vascular bundles are hampered by limited availability as well as the risk of donor site morbidity.^{10,11} Allograft tissue is not ideal due to the risk of disease transmission¹² and the high incidence of graft resorption without adequate bone height maintenance,¹³ especially in alveolar reconstruction. Surgical techniques such as distraction osteogenesis remain in wide use for the reconstruction of large volumetric defects when graft availability is limited.^{14,15} However, the procedure is surgically intensive, requiring prolonged follow-ups for an effective therapy, has associated pain, has the potential for nerve damage if performed in the mandible,^{16,17} and is prone to infectious complications^{18,19} due to extensive use of instruments.

When large volumetric bone defects are stabilized but untreated, they do not heal of their own accord (FIG. 1). Multiple synthetic grafts have been developed to treat large bone defects, with primary focuses on osteoconductivity, their ability to act as a space holder within the large defect to maintain anatomical form, their potential to reduce the local strain to physiological levels, and their infiltration and remodeling capacity by the osteoblasts. Polymeric matrices, gels,^{20,21} ceramic,^{22,23} and composite²⁴⁻²⁷ systems have been widely investigated, and each system has its own unique advantages and drawbacks. These materials have been used as carriers for growth factors, such as clinically-approved bone morphogenetic proteins (BMPs)²⁸ to induce osteogenesis. However, the recommended supra-physiological doses of these clinically-approved BMPs for regenerative applications are several orders of magnitude higher than the physiologic amounts produced by the cells, and these high doses are reported to be associated with exorbitant costs and serious adverse effects including life-threatening cervical swelling, osteoclast activation with transient bone resorption, ectopic bone formation, up-regulation of BMP inhibitors such as noggin, and cyst-like bone void formation.²⁹⁻³² As such, alternative approaches to using the supraphysiologic doses of BMPs will allow greater safety and efficacy in bone regeneration procedures.

Scaffolds are often designed with mechanical properties similar in elasticity and strength to native bone tissue or a material substrate stiffness that allows for proliferation of osteogenic cells. However, the syncing of scaffold strength and degradation makes their use in critical sized defects challenging. There remains a need for additional scaffolds allowing for re-introduced stalled bone regeneration within critical sized defects as well as methods of manufacturing an using the same.

### SUMMARY

This application describes a solution to the problems described above and in part provides scaffolds, particularly bone scaffolds, with localized changes in mechanical properties allowing for re-introduced stalled bone regeneration within critical sized defects. The scaffolds comprise at least one slow-dissolving CaP segment providing prolonged structural support and at least one sacrificial segment comprising a fast-dissolving CaP material that degrades and/or swells over a defined time course in aqueous environments, creating localized compliant regions. Utilizing degradation-induced mechanical stiffness gradients and scaffold degradation ted mechano-transductive pathways (including YAP/TAZ signaling) re-activates bone regeneration activity. Localized changes in mechanical properties in spatial and temporally controlled manner reinduces stalled bone regeneration, promoting uniform vascularized bridging even in central, poorly vascularized regions of large defects without requiring supraphysiologic doses of growth factors such as BMPs.

Certain embodiments are directed to a bone scaffold comprising at least a first segment comprising a slow-dissolving calcium phosphate (CaP) scaffold and at least a second sacrificial segment comprising a fast dissolving CaP scaffold, wherein the at least second sacrificial segment degrades and/or swells when exposed to aqueous fluid environments over a defined time course. Quantitating the dissolution rate of bone scaffolds can involve measuring how quickly the material degrades under controlled conditions, either in vitro (lab-based) or in vivo (within a living system). The process typically focuses on tracking mass loss, structural changes, or chemical release over time. In mass loss measurement (in vitro) the material is weighed to determine the initial mass (Mₒ). The material can be submerged in a physiological solution like simulated body fluid (SBF), phosphate-buffered saline (PBS), or a buffer mimicking blood plasma, often at 37° C. to simulate body temperature. The material is removed periodically, dried, and weighed to determine the mass at (Mₜ) at specific time points (e.g., days, weeks). Dissolution rate can be calculated using (Mₒ−Mₜ)/Mₒ×100% (percent mass loss over time). In certain fast dissolving or sacrificial segments the dissolution rate can be 50, 60, 70, 80, 90 to 100% mass loss over 1, 2, 3, 4, 5, 6, 7, 8, 9, 10, days or weeks or months. In certain slow dissolving or sacrificial segments the dissolution rate and be 1, 10, 20, 30, to 40% mass loss over 1, 2, 3, 4, 5, 6, 7, 8, 9, 10, days or weeks or months or years. Rate can also be expressed as mass lost per unit time (e.g., 0.01, 0.25 0.5, 1, 5, 10 or more mg/day) or change in ion concentration (e.g., mmol/L) per unit time or structural degradation (via imaging) e.g., change in volume (e.g., mm³) or porosity (%) per unit time or decrease in strength (e.g., MPa) per unit time. Dissolution often follows first-order kinetics (exponential decay), so plotting ln(Mₜ/Mₒ) vs. time can yield a rate constant (k).

The bone scaffold can further comprise one or more segments comprised of calcium phosphate ceramics, other ceramics, or metals with interconnected porosity, wherein the bone scaffold has structural continuity between segments. The at least first segment and the at least second sacrificial segment can be porous. In certain aspects the rate of dissolution of the at least second sacrificial segment is faster than the rate of dissolution of the at least first segment. In certain aspects the at least first segment and the at least second sacrificial segment have structural continuity. The at least first segment and the at least second sacrificial segment can have an interconnected porosity. The bone scaffold is produced, at least in part, by 3D printing followed by air-drying of the green body.

In certain aspects the at least second sacrificial segment, at least first segment, or at least second sacrificial segment and the at least first segment contains at least one therapeutic agent. The at least one therapeutic agent can be a drug, a polymer, or a polypeptide. In certain aspects the at least first segment and the at least second sacrificial segment is surrounded completely or partially by a wrap. The wrap can be a collagen wrap or polymer wrap.

Certain embodiments are directed to methods of producing a segmented bone scaffold comprising at least (i) printing at least a first segment using a first ink material having a first fluid dissolution rate when cured, and (ii) printing at least a second sacrificial segment using a second ink material having a second fluid dissolution rate when cured, wherein the bone scaffold is contiguous having structural continuity between the at least first and at least second sacrificial segments. The method further comprises printing is on top of a metal stabilizing unit. The method can further comprise wrapping the bone scaffold. The printing can be continuous, or discontinuous followed by fusion of independently printed segments.

The degradation of sacrificial segments progressively introduces compliant regions that reduce local stiffness, thereby activating mechano-transductive signaling (e.g., YAP/TAZ-mediated pathways) in infiltrating cells such as osteoblasts and stem cells. This re-initiates osteogenesis in stalled, non-healing central regions of critical-size defects, leading to continuous bridging without additional interventions-distinguishing this approach from static or uniformly degrading scaffolds.

In further embodiments, additional segments may incorporate biocompatible metals (e.g., porous titanium) or other ceramics (e.g., bioactive glasses) to enhance overall mechanics or bioactivity while maintaining structural continuity.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Definitions. The following definitions clarify the meaning of specific terms used throughout this patent application. These terms are defined to ensure a clear and consistent understanding of the invention's scope, embodiments, and claims. Unless otherwise specified, the terms used herein have the meanings set forth below.

“Sacrificial segment” or “sacrificial zone” refers to a discrete region or layer within the bone scaffold comprising a fast-dissolving material (such as a calcium phosphate with a higher dissolution rate than adjacent slow-dissolving segments) that is intentionally designed to degrade and/or swell in aqueous physiological environments as the scaffold rehydrates over a defined time course. This degradation creates localized reductions in mechanical stiffness (compliant regions), thereby facilitating mechano-transductive signaling in infiltrating cells and supporting staged transfer of structural and biological roles to newly forming bone tissue. The term encompasses “crumple zone” as used descriptively herein, where degradation mimics controlled compliance changes to re-initiate regeneration in stalled defects.

“Slow-dissolving” or “slow-degrading” (in reference to a calcium phosphate segment or material) refers to a component that exhibits a relatively low rate of mass loss, structural change, or strength reduction when exposed to aqueous fluid environments (such as simulated body fluid or phosphate-buffered saline at 37° C. and physiological pH), typically less than about 40% mass loss over extended periods (e.g., 60 days or more), providing prolonged structural support during early to mid-stages of bone regeneration.

“Fast-dissolving” or “fast-degrading” (in reference to a calcium phosphate segment or material) refers to a component that exhibits a relatively high rate of mass loss, structural change, or strength reduction when exposed to aqueous fluid environments (such as simulated body fluid or phosphate-buffered saline at 37° C. and physiological pH), typically at least about 50% mass loss within shorter periods (e.g., 14 days or less), enabling the creation of localized compliant regions through timed degradation.

“Localized compliant regions” refers to areas within the scaffold where mechanical stiffness is reduced due to the degradation and/or swelling of sacrificial segments, resulting in altered local matrix mechanics (e.g., decreased modulus or increased deformability) that activate mechano-transductive pathways in cells, such as YAP/TAZ signaling, to promote cellular responses including osteogenesis, angiogenesis, and tissue remodeling in non-healing or stalled regions of bone defects.

“Mechano-transductive signaling” or “mechano-transduction” (as further detailed herein) refers to the cellular process of sensing and converting mechanical stimuli (e.g., changes in stiffness, strain, shear, or fluid flow within the scaffold matrix) into biochemical signals that regulate gene expression, cell proliferation, differentiation, and tissue formation. In the context of the present scaffolds, it particularly involves pathways such as YAP/TAZ activation, which mediate the re-initiation of bone regeneration in response to degradation-induced compliance changes.

“Critical-size bone defect” refers to a bone defect of sufficient volume or dimension that it will not heal spontaneously without intervention, due to factors such as inadequate vascularization, excessive interfragmentary motion, or lack of endogenous regenerative capacity, as commonly defined in the art (e.g., defects exceeding certain thresholds in animal models or clinical contexts where bridging fails without grafting or scaffolding).

“Uniform bone bridging” or “continuous bridging” refers to the formation of contiguous, vascularized bone tissue across the entire span of a defect (including central, poorly vascularized regions), as opposed to limited interfacial in-growth or non-union, resulting in restored structural integrity and function.

“Periosteum mimicry” or “periosteum-like layer” refers to the functional role of a wrap (e.g., collagen-based) in guiding intramembranous bone formation, containing regenerative cells and factors within the defect site, preventing soft tissue invasion, and supporting organized ossification similar to the native periosteum.

“Mechano-transduction” refers to the process by which cells sense and respond to mechanical stimuli—such as pressure, tension, or shear forces—by converting these physical signals into biochemical responses that influence cellular behavior and tissue development. In the context of bone repair and scaffolding, it refers to how mechanical forces applied to a healing bone or scaffold stimulate cells (e.g., osteoblasts, osteocytes) to produce bone matrix, adapt to stress, and guide the remodeling process. A fundamental mechanism of biology and biomechanics is that bones strengthen along lines of force, as described by Wolff's Law.

“Bone graft” refers to a medical procedure or material used to replace, repair, or regenerate missing or damaged bone tissue by transplanting or implanting bone or bone-like substances into a defect site. It serves as a scaffold or filler to encourage new bone growth, providing structural support and a framework for osteoblasts to populate and mineralize over time. Bone grafts are commonly employed in cases where natural healing is insufficient-such as large fractures, non-unions (bones that fail to heal), bone loss from trauma or disease (e.g., tumors), or in spinal fusions and dental reconstructions. They can be sourced from the patient's own body (autograft), a donor (allograft), or synthetic materials (alloplast), each with distinct advantages and limitations.

“Scaffold” refers to a technique in regenerative medicine that employs a three-dimensional, biocompatible structure to support the repair and regeneration of damaged or lost bone tissue. These scaffolds are engineered to replicate the natural extracellular matrix of bone, providing a temporary framework where bone-forming cells, such as osteoblasts or stem cells, can adhere, proliferate, and differentiate into functional bone. Typically crafted from materials like natural collagen, synthetic polymers, or ceramics, bone scaffolds are designed with a porous structure to facilitate cell infiltration, nutrient flow, and blood vessel growth—key elements for successful tissue formation. In practice, they may be seeded with cells or growth factors to boost healing and are intended to degrade gradually as new bone replaces them, leaving no permanent foreign material behind.

“Crumple: zone/segment” or “degradation zone/segment” or “sacrificial zone/segment” refers to the specific region or layer within or around the scaffold where the material undergoes controlled breakdown—through processes like hydrolysis, dissolution, or cellular resorption—over time, as it supports bone regeneration. This zone is characterized by its material composition, porosity, and environmental interactions, which dictate the rate and pattern of degradation, ensuring the scaffold gradually transfers mechanical and biological roles to newly forming bone. For instance, in a multi-material scaffold, the degradation zone might be an outer layer of fast-dissolving amorphous calcium phosphate that releases ions early, or a porous core of polylactic acid eroding as osteoblasts infiltrate, designed to align with healing stages like callus formation or remodeling.

“Bone regeneration” refers to the biological process by which new bone tissue is formed to repair, replace, or restore damaged or lost bone, either naturally following an injury like a fracture or with assistance from medical interventions such as scaffolds, grafts, or growth factors. It involves the coordinated activity of cells—primarily osteoblasts (bone-forming cells), osteoclasts (bone-resorbing cells), and mesenchymal stem cells—guided by signaling molecules like bone morphogenetic proteins (BMPs) and mechanical cues via mechano-transduction. The process aims to rebuild bone's structural integrity and function, progressing through stages like inflammation, soft and hard callus formation, and remodeling, or, in engineered scenarios, integrating with artificial frameworks to fill defects. Bone regeneration is critical in treating conditions beyond simple fractures, such as large bone loss from trauma, tumor resection, or congenital defects, and it's a cornerstone of regenerative medicine, leveraging the body's innate capacity to heal while often enhancing it with tailored technologies.

These definitions are intended to be read in their broadest reasonable interpretation consistent with the specification and are not limited to any specific materials, circuit topologies, numerical values of n, or biological examples disclosed herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. (a) Post-injury CT and (b) x-ray radiograph showing limited regeneration in inferior margin after restoration.

FIG. 2. Schematic showing fast degrading zones built into the scaffold for staged re-initiation of bone healing and resulting in continuous bone defect bridging (arrows on left).

FIG. 3. Advantage of using collagen membrane guide with HA scaffold after 4 and 8 weeks in a rabbit radius vs empty defect and autograft. #indicates greater bone volume in scaffold+wrap and autograft compared to scaffold alone (p<0.05).

FIG. 4. Regenerated bone area per μCT slice (proximal to distal interface) showing greater in-growth at interfaces (proximal>distal) than at the defect center. Greater uniformity is observed in autograft and scaffold+wrap.

FIG. 5. Schematic of a Bioplotter showing manufacturing of fast and slow degrading CaP zones on the green body “sacrificial crumple zone” scaffolds.

FIG. 6. Pore morphology impacts bone and vessel development through mechano-transduction (yap1). Pore sizes smaller than 340 μm are required to ensure distribution of vessels across pores (right panels) and promote sustained angiogenesis (hypoxia mediated vegfa) and osteogenesis (bmp2) (Blue arrows indicate increasing time along axis, other axis: pore size compared to fibrin gels in red).⁵⁰

FIG. 7. Formal swelling test of the 65/35 formulation. Graphs showing % swell/time.

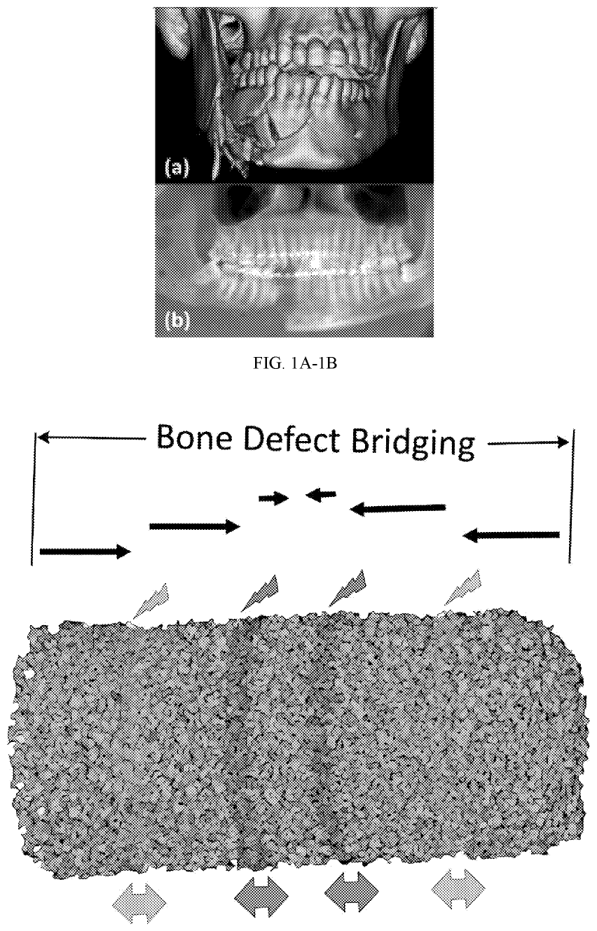

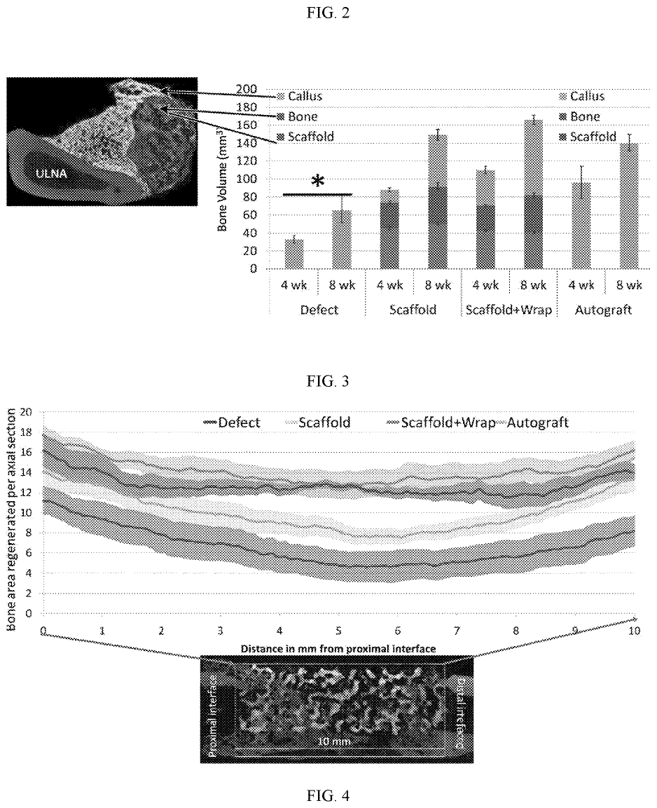

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted as limiting the scope of the disclosure. In addition, one skilled in the art will understand that the following description has broad application, and the discussion of any embodiment is meant only to be an example of that embodiment and not intended to imply that the scope of the disclosure is limited to that embodiment.

Described herein are bone graft scaffolds designed with sacrificial segments (also referred to as crumple zones or degradation zones) to re-introduce stalled bone regeneration within critical-size defects. The scaffolds are fabricated using multi-material 3D printing techniques to create contiguous structures comprising at least one slow-dissolving calcium phosphate (CaP) segment that provides prolonged structural support and at least one fast-dissolving sacrificial segment. The sacrificial segment degrades and/or swells when exposed to aqueous physiological environments over a defined time course. This timed degradation creates localized compliant regions that reduce mechanical stiffness gradients within the scaffold. The resulting changes activate mechano-transductive signaling pathways, including YAP/TAZ-mediated pathways, in infiltrating cells such as mesenchymal stem cells and osteoblasts. This re-initiates osteogenesis in non-healing central regions of large defects, promoting uniform vascularized bone bridging even in poorly vascularized areas, without requiring supraphysiologic doses of growth factors such as bone morphogenetic proteins (BMPs).

### I. BONE SCAFFOLDS

The bone scaffolds of the invention comprise calcium phosphate (CaP) ceramics selected for their biocompatibility and osteoconductivity. In certain embodiments, the scaffold includes at least a first segment comprising a slow-dissolving CaP material (such as hydroxyapatite (HA) or HA-rich blends) and at least a second sacrificial segment comprising a fast-dissolving CaP material (such as β-tricalcium phosphate (β-TCP) or TCP-rich formulations). The differential dissolution rates are achieved by selection of CaP phases, control of porosity, and optimization of ink formulations.

Additional segments may incorporate other biocompatible ceramics (e.g., bioactive glasses) or metals (e.g., porous titanium alloys) with interconnected porosity, provided that structural continuity is maintained between all segments of the scaffold.

The scaffolds are porous with interconnected pores, typically in the range of about 100-500 μm, to enable cell infiltration, nutrient and waste transport, vascular ingrowth, and tissue integration while balancing mechanical integrity.

In certain embodiments, one or more segments, particularly the sacrificial segments, contain at least one therapeutic agent. Suitable agents include drugs, polymers, or polypeptides. A non-limiting example is stromal-derived factor-1 alpha (SDF-1α) encapsulated in poly-lactic-co-glycolic acid (PLGA) microspheres with a poly-ethyleneimine (PEI) coating, immobilized on scaffold surfaces for controlled release to recruit mesenchymal stem cells.

### A. Scaffolding Compositions

Calcium phosphate ceramics are a class of biocompatible materials widely used in bone repair and regeneration, particularly in bone scaffolding and grafting, due to their chemical similarity to the mineral component of natural bone. These ceramics, primarily composed of calcium and phosphate ions, include compounds like hydroxyapatite (HA), tricalcium phosphate (TCP), and biphasic calcium phosphate (BCP—a mix of HA and TCP). Their appeal lies in their osteoconductive properties, meaning they provide a supportive surface for bone cells to grow on, and in some cases, osteoinductive potential, where they stimulate bone formation. These ceramics are often engineered with interconnected pores (50-500 micrometers) to support cell migration, vascularization, and tissue integration. HA offers greater strength, while TCP is weaker but resorbs more readily. Strength varies with porosity-higher porosity aids biology but reduces durability. They bond directly with bone tissue, promoting integration without triggering immune rejection.

In certain embodiments, the scaffold may incorporate additional segments of other biocompatible ceramics (e.g., bioactive glasses) or metals (e.g., porous titanium alloys) with interconnected porosity to enhance mechanical stability, osteoconductivity, or integration while maintaining overall structural continuity across segments.

Creating bone scaffolds that dissolve at different rates involves tailoring their material composition, structure, and degradation mechanisms to match the specific timeline of bone regeneration, ensuring they provide support until new tissue can take over. This can be achieved by using composite materials, such as blending fast-degrading polymers like polyglycolic acid (PGA), which breaks down via hydrolysis in weeks, with slower-degrading options like polylactic acid (PLA) or polycaprolactone (PCL), which persist for months to years, allowing a staged resorption profile. Incorporating calcium phosphate ceramics, like tricalcium phosphate (TCP) for quicker dissolution or hydroxyapatite (HA) for prolonged stability, further fine-tunes the rate—TCP releases ions rapidly to boost early bone growth, while HA lingers to reinforce later stages. Techniques like 3D printing enable precise control over porosity and layer thickness, with higher porosity accelerating degradation by increasing surface area exposed to bodily fluids, while denser zones slow it down. Additionally, embedding hydrolytic enzymes or pH-responsive coatings can trigger degradation on demand, aligning the scaffold's breakdown with healing milestones—say, faster in the initial callus phase and slower during remodeling—offering a custom-fit solution for diverse repair needs.

Bone scaffold degradation or “sacrificial/crumple zones” refers to the controlled breakdown of the scaffold material in the body over time, ideally at a rate that matches the formation of new bone tissue, allowing the scaffold to provide temporary support without leaving permanent foreign material behind. This process is a critical design feature in tissue engineering, ensuring that the scaffold fulfills its role—guiding cell growth, maintaining mechanical stability, and facilitating vascularization—before being replaced entirely by regenerated bone. The degradation rate depends on the material used, the scaffold's porosity, and the physiological environment, with the goal of avoiding both premature collapse (which could destabilize the repair) and prolonged presence (which might hinder full tissue integration). Water molecules break down polymer-based scaffolds (e.g., polylactic acid or polyglycolic acid) into smaller, absorbable fragments like lactic or glycolic acid, which the body metabolizes. Calcium phosphate ceramics like tricalcium phosphate (TCP) dissolve gradually in bodily fluids, releasing calcium and phosphate ions that can contribute to bone mineralization. Osteoclasts or macrophages may resorb certain materials through enzymatic or phagocytic processes, mimicking natural bone remodeling. Factors such as pH, temperature, and local enzyme activity in the implant site influence degradation speed.

In certain embodiments, the slow-dissolving segments comprise crystalline hydroxyapatite (HA) or a 70:30 β-tricalcium phosphate (β-TCP):HA blend, while the fast-dissolving sacrificial segments comprise pure β-TCP or amorphous calcium phosphate-rich formulations. These are prepared as viscous sol-gel inks by mixing the respective CaP powders with 1% v/v ammonium polyacrylate dispersant, 3% v/v N,N-dimethylformamide as drying agent, high molecular weight polyvinyl alcohol, and 1% v/v carboxymethylcellulose binders in distilled water. When cured and air-dried (without high-temperature sintering), the fast-dissolving β-TCP segments exhibit at least 50-80% mass loss within 7-14 days in phosphate-buffered saline (PBS) at 37° C. and pH 7.4, whereas the slow-dissolving HA-rich segments show less than 20-40% mass loss over the same period or up to 60+ days. This differential creates timed localized compliant regions as the sacrificial segments degrade and/or swell, reducing local compressive modulus by 30-70% in those zones while the structural slow-dissolving segments maintain overall continuity and load-bearing capacity during early healing. Avoiding high-temperature sintering (particularly above 800° C.) is preferred because it can densify the material, reduce the differential dissolution rates, limit swelling, and diminish the rapid ion release necessary for mechano-transductive signaling and the crumple-zone effect. In certain applications where higher density is desired, mild thermal processing below temperatures that cause significant densification may be used, but air-drying of the green body is the primary and preferred consolidation method to preserve the functional advantages of the invention.

Herein a layering technique is used creating a strategy of synthesizing a bone graft scaffold with designed “crumple zones” and localized growth factor delivery for stem cell recruitment would enable a single step surgical intervention for bone regeneration: establishing time-delayed mechanical stiffness gradients and incorporating both calcium sources and chemoattractants, to activate the mechano-transductive pathways necessary for sustained vascularized bone regeneration. Ultimately, allowing full-cross section zones of a bone scaffold to degrade at future points of time, in order to leave only the collagen coating as a temporary scaffold within the cellular fibrin plug at a defect and thus re-initiate regeneration. This ability to control local mechanical properties within a structural synthetic bone graft substitute represents a paradigm shift on how scaffold affects bone regeneration, taking into account the effect of mechano-transduction to promote a conducive and robust callus for bone osteogenesis as well as forming a continuous periosteum-like protective layer for intramembranous bone formation.⁵¹ Whether distraction osteogenesis^{52, 53}, reverse dynamization⁵⁴⁻⁵⁶ or compliant plates/fixturing^{57, 58} to increase critical size defect healing via mechano-transduction, current practices need multiple surgical interventions. Creating a biomaterial scaffold with zone/s that turn compliant can potentially engage the same mechano-transductive cues within the scaffold and directly impact the tissue response in situ without further surgical intervention.

In certain aspects the bone scaffold can include chemoattractant properties to enhance bone regeneration through increasing growth factors.

The bone repair process is a multi-stage biological sequence that restores the structural integrity and function of a fractured or damaged bone, relying on the coordinated efforts of various cells, signaling molecules, and the body's natural healing capacity-sometimes aided by interventions like scaffolds or fixation devices. Triggered by injury, it unfolds over distinct phases, typically spanning weeks to months depending on factors like the fracture's severity, location, patient age, and health. It's a dynamic interplay of inflammation, tissue formation, and remodeling, ultimately aiming to return the bone to its pre-injury state or as close as possible.

Chemoattractants are signaling molecules that induce directed cell migration (chemotaxis) toward a specific location, such as a bone scaffold, by creating a concentration gradient that cells follow. In the context of bone repair, they're critical for recruiting key players like stem cells, osteoblasts (bone-forming cells), and endothelial cells (for blood vessel formation) to the scaffold, enhancing its effectiveness in regenerating tissue. These molecules can be naturally produced by the body during injury or intentionally incorporated into scaffolds to boost healing, especially in cases where natural cell recruitment is insufficient, like large defects or compromised tissue environments. Beyond inducing bone formation, BMP-2 and BMP-7 attract mesenchymal stem cells (MSCs) to differentiate into osteoblasts. Vascular endothelial growth factor (VEGF) draws endothelial cells to promote angiogenesis, ensuring the scaffold gets a blood supply. Platelet-derived growth factor (PDGF) recruits MSCs and fibroblasts, aiding early tissue repair.

Stromal derived factor-1 alpha (SDF-1α), a potent CXC chemokine, is expressed during early bone development by MSCs, osteoblasts and fibroblasts⁴⁰ and attracts MSCs as well as circulatory marrow-derived osteoblasts to the injury site usually within 3 days of injury.⁴¹ SDF-1α plays a key role in early bone development since decreased proliferation and impaired osteoblast differentiation have been reported in CXC chemokine-deficient cells and bone tissues, even with BMP2 stimulation, indicating suppressed activation of intracellular BMP receptor-regulated Smads and Erk1/2.⁴² Over time, the callus formed has been reported to act as a biologic immobilizing splint by binding the severed bone sections and inhibiting excessive interfragmentary motion. The soft callus, distinguished by low oxygen tension results in the invasion of blood vessels.^{43,44} Vessel ingrowth leads to calcification as well as remodeling by osteoblasts to lay down osteoid.⁴⁵ This angiogenic-osteogenic coupling⁴⁵ is thus closely regulated by mechano-transductive signaling (transcription factors YAP/TAZ)⁴⁶⁻⁴⁹, and our recent works₅₀ has shown that scaffold pore size and thus matrix mechanics within pores accentuates this development of capillary networks within the bone stroma.

Therapeutic agents such as SDF-1α are optionally incorporated via PLGA microspheres with PEI coating, immobilized on scaffold surfaces (including sacrificial zones) for controlled release over 2-30 days, as quantified by ELISA. The differential degradation ensures that chemoattractant release coincides with creation of compliant regions, enhancing MSC recruitment and vascular ingrowth in central defect areas. All embodiments maintain structural continuity between segments through direct printing or fusion, with interconnected porosity permitting fluid permeation and cell infiltration. These parameters were iteratively optimized using non-working formulations (e.g., sintered versions losing crumple efficiency or high-Bloom gelatin causing poor printability) to define workable boundaries, enabling one of ordinary skill to practice the invention across the claimed scope without undue experimentation.

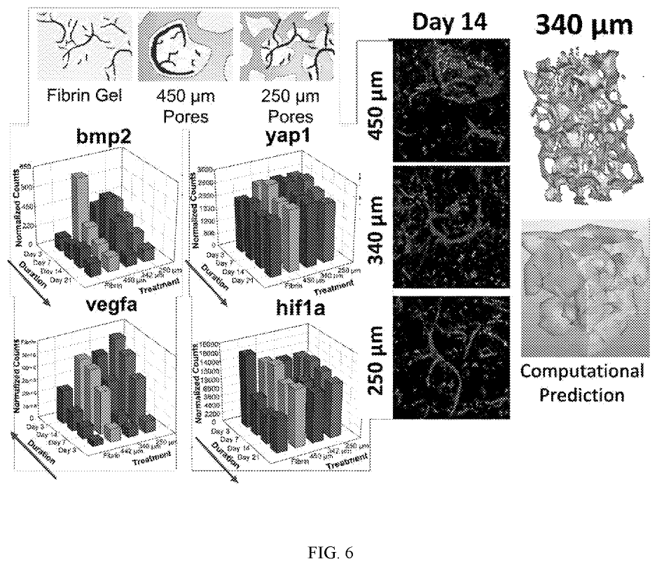

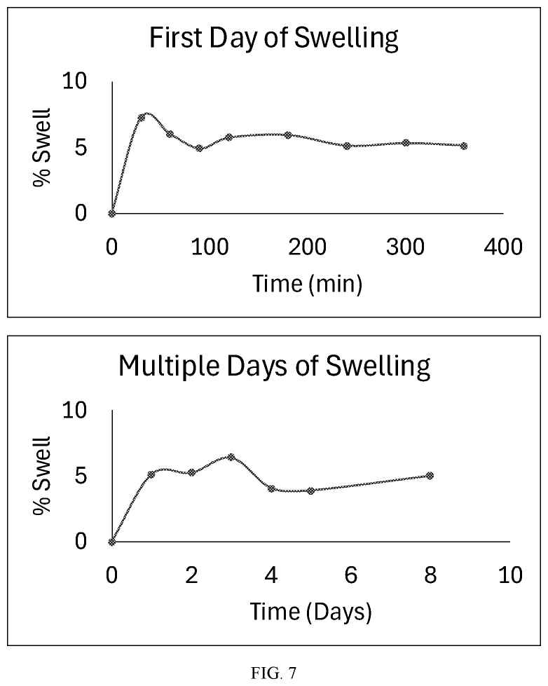

In certain aspects SDF-1α can be encapsulated in poly-lactic-co-glycolic acid (PLGA) microspheres (MS) with an outer poly-ethyleneimine (PEI) coating for MS stabilization and growth factor release control. As demonstrated by our group,⁶⁹ PLGA polymer with a PLA:PGA molar ratio of 75:25 will be mixed with different SDF-1α dosages (R&D Systems, MN) and dissolved in a co-solvent containing dichloromethane and ethanol, followed by emulsification. This solution will then be mixed with a 0.5% polyvinyl alcohol (PVA) and stirred to allow solvent evaporation. The hardened MS will be collected by centrifugation, washed with distilled water, and lyophilized using a freeze dryer. Immobilization of loaded PLGA MS on scaffold surfaces will be performed in a three-step process.⁶⁹ The MS surfaces will be radiofrequency plasma glow-discharged in an oxygen-filled chamber followed by dispersing the MS in the positively-charged PEI solution (5 mL, 0.05 wt. %) for 12 hour at pH 7.0. PEI-coated MS can then be rinsed with distilled water to remove excess PEI followed by lyophilizing in a freeze dryer. In the final step, 30 mg of PEI-coated MS dispersed in distilled water will be added to the porous scaffolds, followed by gentle shaking. Unimmobilized MS will be removed by washing and the scaffolds dried overnight at room temperature. Scaffolds will then be coated with a 4% (w:v) collagen in 0.05M acetic acid solution by immersion and drainage. SDF-1α loading, release kinetics and retention of activity will be measured by ELISA kits (R&D Systems) prior to and post collagen coating by placing the scaffold in PBS at a physiologic pH and collecting the PBS over a 30 day period. Our preliminary study shows the ability to load dexamethasone (DEX)-tethered MS on scaffold surfaces (FIG. 6), exhibiting delayed release compared to the non-tethered DEX (control).⁶⁹

Bone scaffolds with tailored dissolution rates can be crafted from amorphous calcium phosphate (ACP) and crystalline calcium phosphate (CCP), such as tricalcium phosphate (TCP) or hydroxyapatite (HA), by exploiting their differing degradation profiles to support the phased process of bone regeneration. ACP, with its non-crystalline, unstable structure, dissolves rapidly in weeks through hydrolysis or cellular resorption, releasing calcium and phosphate ions to jumpstart early bone formation during the soft callus stage. In contrast, TCP, a crystalline form, degrades more gradually over months via dissolution and osteoclast activity, providing mid-term stability as the hard callus develops, while HA, another crystalline variant, breaks down very slowly over years due to its bone-like stability, sustaining support through remodeling. Using techniques like 3D printing, these materials can be combined into a composite scaffold—say, an ACP-rich surface for quick ion release paired with a TCP or HA core for durability—or layered with varying porosity, where higher porosity accelerates ACP's breakdown and denser zones slow HA's, creating a controlled degradation gradient that aligns with healing, from rapid initial dissolution to prolonged structural reinforcement.

Amorphous calcium phosphate (ACP) and crystalline calcium phosphate (CCP) differ significantly in their dissolution rates due to their structural differences, as is described here. Amorphous Calcium Phosphate (ACP) lacks a long-range, ordered crystalline structure, making it more disordered and less stable. ACP dissolves faster than its crystalline counterparts. Its amorphous nature results in a higher surface energy and reactivity, leading to quicker breakdown in aqueous environments. Studies suggest that ACP can dissolve rapidly under physiological conditions (e.g., pH 7.4), often transforming into more stable phases like hydroxyapatite over time. The rate is highly sensitive to pH, temperature, and the presence of ions (e.g., HPO₄²⁻, Ca²⁺). In acidic conditions (e.g., pH<6), dissolution accelerates significantly.

Crystalline Calcium Phosphate (CCP), such as hydroxyapatite (Ca₁₀(PO₄)₆ (OH)₂) or tricalcium phosphate (Ca₃ (PO₄)₂), has a well-defined, ordered lattice structure, making it more thermodynamically stable. CCP dissolves much more slowly than ACP. The strong ionic bonds and organized crystal lattice resist breakdown, requiring more energy or harsher conditions (e.g., lower pH) to dissolve. For example, hydroxyapatite is known for its low solubility product (Ksp≈10⁻⁵⁸), indicating minimal dissolution in neutral or basic environments. Dissolution increases in acidic conditions (e.g., pH<5) or with mechanical stress, but it remains slower than ACP. The specific crystalline form (e.g., hydroxyapatite vs. brushite) also affects the rate.

Speed: ACP dissolves faster (often within minutes to hours under physiological conditions) compared to CCP (which may take days or remain stable indefinitely in neutral pH). Stability: ACP is metastable and prone to spontaneous conversion to crystalline forms, while CCP is stable unless exposed to extreme conditions. Applications: ACP's rapid dissolution is leveraged in biomaterials for quick ion release (e.g., in bone repair), while CCP's slow dissolution suits long-term stability (e.g., in dental enamel or implants).

Other scaffold materials may include the following: Hydroxyapatite (HA) is chemically identical to bone mineral (Ca₁₀(PO₄)₆(OH)₂), HA is highly stable and biocompatible but degrades very slowly, making it ideal for permanent implants or long-term scaffolding. Tricalcium phosphate (TCP) has the formula of Ca₃(PO₄)₂, TCP is more soluble than HA, degrading faster in the body and releasing calcium and phosphate ions that can aid bone remineralization—perfect for temporary scaffolds. Biphasic calcium phosphate (BCP) is blend of HA and TCP, BCP balances stability and degradation, allowing tailored resorption rates for specific applications.

Calcium phosphate ceramics are a cornerstone of modern orthopedics and dentistry, bridging the gap between synthetic materials and biological repair although they have limitations associated. Overall, they lack the toughness of natural bone, limiting use in load-bearing areas unless combined with polymers or metals. HA can persist too long, while TCP may dissolve too quickly for some repairs. Achieving the right porosity and strength requires precise manufacturing (e.g., 3D printing).

Metals used for bone scaffolding are valued for their exceptional mechanical strength and durability, making them suitable for load-bearing applications in bone repair, though they are less common than ceramics or polymers due to their limited biodegradability. Titanium and its alloys, such as Ti-6Al-4V, dominate this category because of their biocompatibility, corrosion resistance, and ability to integrate with bone tissue through osseointegration—a process where bone cells grow directly onto the metal surface. Stainless steel and cobalt-chromium alloys are also used, offering high toughness and wear resistance, particularly in orthopedic implants like hip or knee replacements that may incorporate scaffold-like porous zones. To enhance biological performance, these metals are often 3D-printed or processed into porous structures with controlled porosity (e.g., 100-500 micrometer pores) to allow cell infiltration and vascularization, though they typically remain permanent rather than degrading like ideal scaffolds. Surface modifications, such as coating with hydroxyapatite or growth factors, further improve their bioactivity, bridging the gap between metal's mechanical advantages and the biological needs of bone regeneration-though their non-resorbable nature often relegates them to hybrid designs alongside degradable materials.

### B. Porosity

Porosity is the presence of interconnected voids or pores within the scaffold structure, a critical design feature that significantly influences its performance in bone repair and regeneration. These pores mimic the natural porous architecture of bone, such as the trabecular (spongy) bone, and are essential for facilitating cell infiltration, nutrient diffusion, waste removal, and vascularization-all key to successful bone tissue growth. The degree of porosity, pore size, and interconnectivity are carefully engineered to balance biological functionality with mechanical strength, making porosity a cornerstone of effective scaffold design. Typically 100-500 micrometers is ideal for bone regeneration. Pores must be large enough for osteoblasts (bone-forming cells) and blood vessels to penetrate but small enough to maintain structural integrity. Scaffolds often have 60-90% porosity, meaning 60-90% of their volume is void space. Higher porosity boosts biological activity (more space for cells and vessels) but reduces mechanical strength, while lower porosity strengthens the scaffold but may hinder tissue integration. Pores must be interconnected, not isolated, to allow continuous pathways for cells, nutrients, and blood vessels. Isolated pores trap cells and stunt growth. Porous structures provide space for stem cells or osteoblasts to migrate and proliferate, forming new bone tissue. Blood vessel growth (angiogenesis) into the scaffold delivers oxygen and nutrients, critical for large-scale repairs where diffusion alone isn't enough. Open pores ensure cells deep within the scaffold survive by enabling the flow of nutrients in and waste out. Porosity influences how quickly a scaffold breaks down—higher porosity often accelerates resorption as more surface area is exposed to bodily fluids.

### C. Therapeutic Agents

In certain aspects the bone scaffolds can include various therapeutic agents. Therapeutic agents beyond chemoattractants and growth factors, encompass a range of bioactive substances designed to address infection, inflammation, resorption, and cellular integration, tailoring the scaffold's role in bone repair. Antibiotics like gentamicin, vancomycin, or tetracycline can be embedded to prevent or treat infections such as osteomyelitis, particularly in open fractures, releasing locally to minimize systemic side effects while tackling bacteria that thrive in porous structures. Anti-inflammatory drugs, such as ibuprofen or dexamethasone, can be incorporated to dampen excessive inflammation that could delay healing or provoke scaffold rejection, delivering relief directly at the site. Calcium and phosphate ions, naturally released from degrading calcium phosphate ceramics like tricalcium phosphate or hydroxyapatite, act as therapeutic agents by enhancing osteoblast activity and mineralization, supporting bone formation without additional drugs. Bisphosphonates like alendronate or zoledronate can be added to inhibit osteoclast-driven bone resorption, preserving scaffold integrity and new bone in conditions like osteoporosis. Peptides such as RGD motifs or small molecules like strontium ranelate can be included to improve cell adhesion or stimulate osteogenesis, offering simpler, cost-effective alternatives to proteins. These agents are integrated through methods like polymer entrapment, surface coating, or 3D-printed encapsulation, ensuring controlled release that aligns with the healing process, making scaffolds versatile tools for complex bone regeneration challenges.

### II. FABRICATION BY 3D PRINTING

In certain aspects the segmented bone scaffold can be fabricated by 3D printing. Ultimately allowing for the scaffold to be tailored directly to the needs of a specific subject or patient.

3D printing bone scaffolds is a transformative approach in tissue engineering that leverages additive manufacturing to create precise, patient-specific structures for bone repair and regeneration. By using digital models-often derived from medical imaging like CT or MRI scans—3D printing allows for the fabrication of scaffolds with tailored shapes, sizes, and internal architectures that closely match a patient's anatomy and the specific needs of the injury. This technology has revolutionized bone scaffolding by enabling the use of materials like calcium phosphate ceramics, biocompatible polymers, and even bioinks containing living cells, offering unprecedented control over the scaffold's properties and performance.

During the design process, a digital 3D model is created, specifying the scaffold's geometry, porosity (e.g., pore sizes of 100-500 micrometers for cell growth), and mechanical requirements. Printing includes material selection and printing technique selection. During post-processing scaffolds may be dried or sterilized before implantation.

3D printing with two or more materials, often referred to as multi-material or dual-extrusion printing, involves using multiple material types simultaneously in a single print job to create objects with varied properties-such as degradation rates. This techniqe typically relies on Fused Deposition Modeling (FDM) printers equipped with multiple extruders (e.g., dual or triple heads) or a single nozzle with a filament-switching system, allowing seamless transitions between materials layer-by-layer or within the same layer. For bone scaffolds, this approach enables the fabrication of complex structures with tailored functionalities, combining materials like fast-dissolving polymers with durable or bioactive ones to match the dynamic needs of bone regeneration.

In certain embodiments the segmented scaffolds are fabricated by continuous or multi-head 3D bioplotting on a Bioplotter equipped with multiple print heads, allowing simultaneous deposition of fast-dissolving (β-TCP-rich) and slow-dissolving (HA or β-TCP:HA blend) inks in layered or zoned architectures. Printing may occur atop an optional metal stabilizing unit if needed for large defects. Overhanging geometries are supported by printed paraffin, which is fully removed during air-drying without requiring a furnace step. Post-printing, the green body is air-dried at room temperature or under mild conditions, resulting in structural continuity and interconnected porosity (mean pore interconnection ~340 μm) across segments. The entire scaffold is then dip-coated twice with 4% (w/v) rat tail collagen Type I in 0.05 M acetic acid to deposit thin films that enhance toughness, mimic periosteum, and further modulate surface degradation. Sacrificial zones are preferably implemented as discrete repeating layers of 0.25-0.5 mm thickness spaced 1-2 mm apart along the longitudinal axis. This configuration ensures progressive introduction of compliant regions as the fast segments degrade/swell over a defined 7-21 day course in aqueous environments, while slow segments provide prolonged support, collectively activating YAP/TAZ pathways without collapse of the overall construct. Air-drying the green body is critical to preserving the differential swelling and dissolution behavior between segments that enables the mechano-transductive crumple zones.

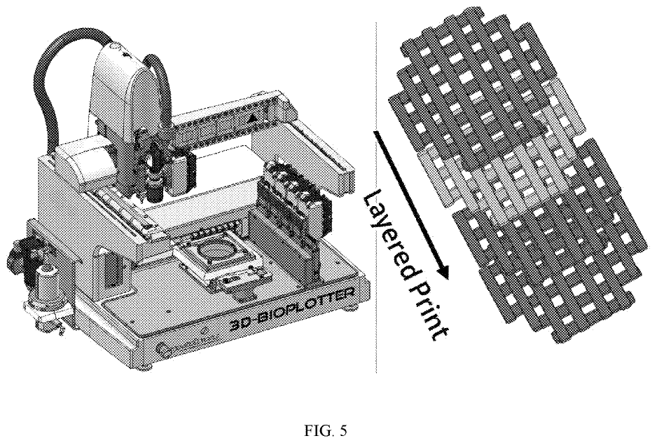

In certain aspects scaffold fabrication can go as follows. Porous scaffolds, as prepared by previously reported template coating, 34 with mean porous interconnection size of 340 μm will be scanned using micro-computed tomography (uCT) and digitized to create a stereolithography (STL) file using Mimics (Materialise, Belgium). With the STL file and using a 3D printer capable of high spatial resolution (Bioplotter, EnvisionTEC, MI), scaffolds with “sacrificial crumple zones” will be fabricated. A green body using a viscous sol-gel form of either fast degrading CaP (beta-tricalcium phosphate, βTCP), a slower degrading CaP (a 70:30 blend of βTCP:HA) and a relatively slow degrading CaP (100% crystalline HA) will be printed. As previously described,48 different CaP sol-gel slurries, used as inks, will be constituted by βTCP, HA or a mixture of βTCP and HA mixed with 1% v/v ammonium polyacrylate dispersant and 3% v/v N,N-dimethylformamide drying agent; combined with high molecular weight polyvinyl alcohol and 1% v/v carboxymethylcellulose binders in distilled water65. The BioPlotter, equipped with multiple print heads will be used to print layer-by-layer of slow degrading CaP scaffold interspersed with fast degrading zones of βTCP and more centrally slower degrading bands of βTCP:HA (FIG. 5). Since printed architectures have overhanging geometries in the individual layers, a paraffin support material will be printed in the pores during fabrication. The green body is then air-dried at room temperature or under mild conditions. During the process, the coalescence of HA and βTCP grains will occur in their independent layers and the paraffin will be completely removed. The entire scaffold will be dip-coated with 4% (w:v) rat tail collagen I in 0.05M acetic acid solution by immersion and drainage to deposit two successive thin films of collagen on the scaffold surface. In this study, sacrificial crumple zones will be prepared in a two-layer thicknesses (0.25 and 0.5 mm) and spaced at two different zone spacings (1 mm and 2 mm) within a cylinder 6 mm tall and 3 mm in diameter. All scaffolds will be sterilized by ethylene oxide prior to testing.

### A. Bone Scaffold Inks

In 3D printing of bone scaffolds, the “ink” refers to the material or bioink used to create the scaffold structure. These inks are designed to mimic the properties of bone, such as mechanical strength, porosity, and biocompatibility, while supporting cell growth and tissue regeneration. Below are some examples of inks commonly used or researched for printing bone scaffolds:

Hydrogel-Based Bioinks with Bioceramics. One example is Gelatin Methacrylamide (GelMA) combined with Hydroxyapatite (HAp). GelMA is a photocrosslinkable hydrogel derived from gelatin, often mixed with hydroxyapatite nanoparticles (n-HAp), a key mineral component of natural bone. This ink provides a biocompatible matrix for cell encapsulation and promotes osteogenic differentiation due to HAp's osteoconductive properties. It is often used in digital light processing (DLP) or extrusion-based printing for high-precision scaffolds.

Calcium Phosphate-Based Inks. One example is β-Tricalcium Phosphate (β-TCP) with Polymeric Binders. β-TCP, a biodegradable ceramic similar to bone mineral, can be mixed with binders like polyethyleneimine or Pluronic® F-127 to form a printable colloidal gel. This ink is commonly used in direct ink writing (DIW) or inkjet printing, offering excellent mechanical strength and bioresorbability for load-bearing applications.

Polymer-Ceramic Composite Inks. One example is Polylactic Acid (PLA) with Nano-Hydroxyapatite (n-HA). PLA, a biodegradable synthetic polymer, is blended with n-HA to enhance mechanical properties and bioactivity. This ink is typically used in fused deposition modeling (FDM) to create porous scaffolds that mimic the organic and inorganic components of bone, supporting cell adhesion and proliferation.

Natural Polymer-Based Inks. One example is Sodium Alginate with Cuttlebone and Gelatin. Sodium alginate, a seaweed-derived polysaccharide, can be combined with cuttlebone (a calcium carbonate source) and fish gelatin to form a paste-like ink. This bioinspired formulation mimics bone's extracellular matrix and is used in extrusion-based printing, promoting biomineralization and cell growth.

Photocrosslinkable Nanocomposite Inks. One example is Nano-Hydroxyapatite Methacrylate (nHAMA) with Poly(Propylene Glycol) Dimethacrylate (PmLnDMA). This ink integrates nHAMA (a modified hydroxyapatite) with a synthetic polymer matrix that crosslinks under light exposure. It offers tunable mechanical strength (up to 400 MPa compressive modulus) and is suitable for extrusion-based printing, providing scaffolds with high osteogenic potential and the ability to release growth factors like BMP-2.

Bioactive Glass Inks. One example is κP53B Glass with Pluronic® F-127. Bioactive glass (e.g., 6P53B composition: 52.7% SiO₂, 18% CaO, 6% P₂O₅, etc.) is dispersed in a hydrogel like Pluronic® F-127 to create a shear-thinning ink. Used in direct ink writing, this ink produces scaffolds with compressive strength comparable to cortical bone (around 136 MPa) and high porosity, ideal for bone defect repair.

Collagen-Based Bioinks. One example is Collagen with Decellularized Extracellular Matrix (ECM) and Silk Fibroin. Collagen, a primary organic component of bone, is mixed with ECM and silk fibroin to enhance mechanical stability and bioactivity. This ink is used in low-temperature extrusion printing to create micro/nanoporous scaffolds that support osteogenic differentiation and tissue integration.

Gelatin. Gelatin serves as a critical component in the CaP sol-gel inks, modulating rheological properties, swelling behavior, degradation kinetics, and initial compliance of the green-body scaffolds. Bloom strength, a measure of gel rigidity directly correlated with average molecular weight and crosslinking potential, influences mesh size and crosslink density within the hydrogel network—higher Bloom values generally increase stiffness and reduce swelling, while lower Bloom gelatin improves extrusion flow and printability on the multi-head Bioplotter. Type A gelatin (acid-processed, isoelectric point approximately pH 7-9) and Type B gelatin (alkaline-processed, isoelectric point approximately pH 4-6) differ in free carboxyl group content and charge behavior; matching the slightly acidic to neutral pH of the bioink (containing HA, β-TCP, and carboxymethylcellulose) to the gelatin's isoelectric point minimizes electrostatic repulsion between chains, thereby enhancing physical crosslinking without additional chemical agents. Gelatin concentration further tunes these properties-higher concentrations increase viscosity, crosslink density, and mechanical integrity but can reduce the rapid swelling/degradation differential essential for sacrificial segments, whereas optimized lower concentrations (as in the 65/35 HA/gelatin blends) balance printability with controlled water uptake (~2-5% dimensional change) and ion release while preserving the differential dissolution rates between fast-dissolving (β-TCP-rich) and slow-dissolving (HA-rich) zones after air-drying.

Bloom strength, a standard measure of gelatin gel rigidity (expressed in Bloom grams), reflects the average molecular weight and the ability of gelatin chains to form physical crosslinks through hydrogen bonding and electrostatic interactions. In the present invention, gelatin Bloom strength is deliberately varied to balance printability, swelling behavior, degradation kinetics, and the creation of localized compliant regions in the sacrificial segments. High-Bloom gelatin (typically 200-300 Bloom) provides higher crosslink density, reduced mesh size, and greater initial mechanical stiffness, whereas low-Bloom gelatin (typically 50-150 Bloom) enhances extrusion flow and facilitates rapid swelling upon rehydration. Working formulations of the CaP sol-gel inks utilized blends of Type A gelatin incorporating both high-Bloom (220-280 Bloom) and low-Bloom (80-120 Bloom) grades at overall gelatin concentrations ranging from 10% to 35% (w/w relative to HA), with the optimized 65/35 HA/gelatin ratio employing a mixture of high- and low-Bloom Type A gelatin to achieve reliable multi-head Bioplotter printability, modest swelling (~2-5% dimensional change), and the differential dissolution rates necessary for timed introduction of compliant crumple zones without compromising structural continuity after air-drying.

Gelatin is incorporated into the bioink formulations to modulate rheological properties, swelling behavior, and degradation kinetics that are critical to the function of the sacrificial segments. During development in the inventors' laboratory, gelatin was varied by molecular weight, Bloom strength, and processing method (Type A versus Type B) to tailor the hydrogel network. This work is shown in Example 2. Higher Bloom gelatin decreases mesh size and increases crosslink density, while different gelatin types provide access to distinct isoelectric points (pI). By matching the bioink pH as closely as possible to the gelatin's pI, electrostatic repulsive forces between chains are minimized, thereby promoting stronger physical crosslinking. Swelling studies in deionized water or PBS showed that the resulting hydrogels swelled only slightly (~2-5% dimensional change) and maintained a stiff, rubbery consistency upon probing, even after 2-4 hours, without rapid disintegration or residue formation.

In particular, the 65/35 HA/gelatin formulation (using blends of high and low Bloom Type A gelatin at reduced overall gelatin concentration) exhibited significantly improved swelling and degradation properties compared with other ratios tested. Higher gelatin content or Bloom strength increases crosslink density and slows degradation rate, while lower Bloom Type A gelatin improves extrusion without causing excessive stiffness. UV crosslinking attempts at 254 nm provided minimal additional benefit for these formulations. These gelatin-stabilized inks enable reliable multi-head Bioplotter extrusion of contiguous fast- and slow-dissolving CaP zones without delamination. The preserved gelatin phase after air-drying further contributes to initial compliance and controlled Ca²⁺/PO₄³⁻ ion release, supporting early cell recruitment and mechano-transductive signaling without compromising long-term scaffold integrity.

Working formulations identified through iterative testing (e.g., 80/20 and 90/10 HA/gelatin ratios with high/low Bloom blends) confirmed that reducing gelatin concentration and avoiding prolonged high-temperature exposure during preparation preserves printability and the desired differential dissolution between segments. Air-drying the green body at room temperature or under mild conditions (e.g.,

These inks vary based on the printing technique (e.g., inkjet, extrusion, DLP) and the desired scaffold properties, such as mechanical strength, degradation rate, or cellular response. These formulations can be tailored by adjusting the ratios of components or adding bioactive agents (e.g., growth factors) to optimize performance for specific bone regeneration applications.

### B. Scaffold Wrapping

In certain embodiments, the bone scaffold is surrounded completely or partially by a biocompatible wrap configured to support tissue integration, prevent soft tissue invasion, and guide regeneration. Preferred wraps include collagen membranes that mimic the periosteum, promoting intramembranous bone formation, enhancing periosteal remodeling, and improving uniform bone bridging across the defect (as demonstrated in rabbit radius models where collagen-wrapped HA scaffolds showed significantly greater bone volume and interfacial in-growth compared to unwrapped scaffolds). Other biocompatible wraps (e.g., polymer-based) may be employed. The wrap may be applied by dip-coating, immersion, or other methods post-fabrication.

Surface coating can include thin layers of hydroxyapatite, collagen, or calcium phosphate ceramics applied via dipping, spraying, or plasma deposition. It Enhances bioactivity and osteoconductivity, encouraging bone cells to grow onto the scaffold surface, while potentially slowing degradation of the core material (e.g., a PLA scaffold coated with HA).

Hydrogel Encapsulation can include Gelatin, alginate, or hyaluronic acid hydrogels encasing the scaffold. It acts as a soft, hydrated “wrap” to deliver therapeutic agents (e.g., antibiotics like vancomycin) or mimic the extracellular matrix, aiding cell infiltration and vascularization.

Polymer sheathing can include biodegradable polymers like PLA or PCL extruded or molded around the scaffold. It Provides a protective outer layer that degrades at a different rate than the core, offering staged support or controlled release of embedded drugs (e.g., bisphosphonates).

Membrane wrapping can include collagen membranes, synthetic meshes, or periosteum-like sheets (the bone's natural outer layer). It mimics the periosteum to guide tissue growth, retain cells within the scaffold, or prevent soft tissue invasion into the repair site.

Cell-seeded layers can include pre-seeded layers of mesenchymal stem cells or osteoblasts in a matrix (e.g., fibrin). It wraps the scaffold with living cells to jumpstart regeneration and improve integration with native bone.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Scaffold Fabrication-Porous scaffolds, as prepared by previously reported template coating, 34 with mean porous interconnection size of 340 μm will be scanned using micro-computed tomography (uCT) and digitized to create a stereolithography (STL) file using Mimics (Materialise, Belgium). With the STL file and using a 3D printer capable of high spatial resolution (Bioplotter, EnvisionTEC, MI), scaffolds with “sacrificial crumple zones” will be fabricated. A green body using a viscous sol-gel form of either fast degrading CaP (beta-tricalcium phosphate, βTCP), a slower degrading CaP (a 70:30 blend of βTCP:HA) and a relatively slow degrading CaP (100% crystalline HA) will be printed. As previously described, 48 different CaP sol-gel slurries, used as inks, will be constituted by βTCP, HA or a mixture of βTCP and HA mixed with 1% v/v ammonium polyacrylate dispersant and 3% v/v N,N-dimethylformamide drying agent; combined with high molecular weight polyvinyl alcohol and 1% v/v carboxymethylcellulose binders in distilled water65. The BioPlotter, equipped with multiple print heads will be used to print layer-by-layer of slow degrading CaP scaffold interspersed with fast degrading zones of βTCP and more centrally slower degrading bands of βTCP:HA (FIG. 5). Since architectures being printed have overhanging geometries in the individual layers, a paraffin support material will be printed in the pores during fabrication. The green body will then be air-dried at room temperature or under mild conditions. During the process, the coalescence of HA and βTCP grains will occur in their independent layers and the paraffin will be completely removed. The entire scaffold will be dip-coated with 4% (w:v) rat tail collagen I in 0.05M acetic acid solution by immersion and drainage to deposit two successive thin films of collagen on the scaffold surface. In this study, sacrificial crumple zones will be prepared in two layer thicknesses (0.25 and 0.5 mm), and spaced at two different zone spacings (1 mm and 2 mm) within a cylinder 6 mm tall and 3 mm in diameter. All scaffolds will be sterilized by ethylene oxide prior to testing.

Mechanical Properties—The initial scaffold's compression strength and modulus will be measured in a hydrated state using an MTS Insight 5 in displacement control mode at a constant strain rate of 0.125 mm/min. Given the intra-group variation observed in previous studies, 12 samples/group will be used for the compression and diametral tensile testing to ensure statistical power of 0.9. Conforming to the ASTM D695 compression testing of 2:1 aspect ratio, cylindrical scaffolds (12×6 mm) will be prepared. All scaffolds will be mounted in aluminum end caps to minimize edge artifacts prior to testing and will be equilibrated to 37° C. in distilled water for 2 hours.

Physical Properties-Degradation, porosity, fluid permeability and architecture will be measured as previously reported by us⁶⁵. Since the wound healing environment is acidic, scaffold degradation will also be assessed under a low pH condition.⁶⁸ 10 scaffolds/group will be weighed, placed individually in 48-well plate followed by immersion in 1 ml sterile phosphate buffered saline (PBS), and incubated at 37° C., 5% CO2, and at pH of 7.4 or 6.0. Solution will be changed daily and Ca²⁺ released into the PBS will be measured using an o-crestopthalein based colorimetric assay (Pointe Scientific, MI) for up to 56 days. Samples will be removed every 7 days, dried and weighed to determine weight loss calculated. Energy dispersive x-ray spectroscopy and elemental mapping will be used to determine the calcium:phosphorus ratio and distribution of Ca²⁺ and phosphorus ions in different scaffold zones. Using a Micromeritics AccuPyc 1340 helium pycnometry, scaffold porosity (6 scaffolds/group) will be determined. Scaffold weight and theoretical specific gravity will be used as controls for pycnometry measurement accuracy. Fluid permeability (6 scaffolds/group) will be measured using a custom flow apparatus consisting of a distilled water reservoir that feeds into a sample chamber opened to the atmosphere. Using Darcy's Law, permeability is defined as k=mμl/AcsρΔP, where k-permeability; m=mass flow rate; μ=fluid viscosity; L=scaffold length; Acs=mean cross sectional area; ρ=fluid density; and ΔP=pressure drop across the scaffold. Permeability will then be computed by measuring the volume of water collected over four different lengths of time through the scaffold. Scaffold architecture (6 scaffolds/group) will be measured using a SkyScan 1076 μCT at 8.87 μm spatial resolution. Bone volume ratio, bone surface to total volume ratio, bone surface density, trabecular pattern factor, structural model index, trabecular thickness, trabecular number and trabecular spacing will be computed and will be used to determine accuracy and repeatability of fabrication⁶⁵.

Chemoattractant Incorporation-After characterization, SDF-1α will be encapsulated in poly-lactic-co-glycolic acid (PLGA) microspheres (MS) with an outer poly-ethyleneimine (PEI) coating for MS stabilization and growth factor release control. As demonstrated by our group,⁶⁹ PLGA polymer with a PLA:PGA molar ratio of 75:25 will be mixed with different SDF-1a dosages (R&D Systems, MN) and dissolved in a co-solvent containing dichloromethane and ethanol, followed by emulsification. This solution will then be mixed with a 0.5% polyvinyl alcohol (PVA) and stirred to allow solvent evaporation. The hardened MS will be collected by centrifugation, washed with distilled water, and lyophilized using a freeze dryer. Immobilization of loaded PLGA MS on scaffold surfaces will be performed in a three-step process.⁶⁹ The MS surfaces will be radiofrequency plasma glow-discharged in an oxygen-filled chamber followed by dispersing the MS in the positively-charged PEI solution (5 mL, 0.05 wt. %) for 12 hour at pH 7.0. PEI-coated MS will then be rinsed with distilled water to remove excess PEI followed by lyophilizing in a freeze dryer. In the final step, 30 mg of PEI-coated MS dispersed in distilled water will be added to the porous scaffolds, followed by gentle shaking. Unimmobilized MS will be removed by washing and the scaffolds dried overnight at room temperature. Scaffolds will then be coated with a 4% (w:v) collagen in 0.05M acetic acid solution by immersion and drainage. SDF-1α loading, release kinetics and retention of activity will be measured by ELISA kits (R&D Systems) prior to and post collagen coating by placing the scaffold in PBS at a physiologic pH and collecting the PBS over a 30 day period. Our preliminary study shows the ability to load dexamethasone (DEX)-tethered MS on scaffold surfaces (FIG. 6), exhibiting delayed releasecompared to the non-tethered DEX (control).⁶⁹

In vitro Effect on Cell Migration—The effect of the SDF-1α incorporated “sacrificial crumple zone” CaP scaffolds on growth factor release and cellular migration will be evaluated. To ensure that SDF-1α released from the MS retains bioactivity, the VEGF and bFGF production (measured by ELISA kits) and micro-capillary formation capability of the human microvascular endothelial cell line in fibrin microgels70 will be exposed to various concentrations SDF-1α and MS-released SDF-1α to ascertain if the recruitment and pro-angiogenic function of SDF-1α is retained after encapsulation and release. The migration behavior of mesenchymal stem cells (MSC, ATCC PCS-500-12) and hematopoietic stem cells (HSC, CD34+ fraction from human bone marrow, PCS-800-012) will also be evaluated using chemotaxis inserts with 8 μm polycarbonate inserts separating upper and lower chambers of a multi-well chemotaxis assay plate.⁷¹ This would allow for the evaluation of the distinct progenitor populations and a mechanistic explanation to the relative sensitivity of marrow and circulatory stromal cells to the chemokine. The cells would then be GFP labeled and placed in the upper chamber of the chemotaxis assay plate. Varying concentrations of SDF-1α (10, 50, 100, 250, 500 ng/ml)⁷² will be placed in 25 μl in the lower chamber. The factors will be directly applied in the media to evaluate dose response behavior in the migration of cells. Based on the dose response study, the appropriate dose of SDF-1α (inducing greatest chemotactic migration) will be either be directly loaded into the collagen I coating on the scaffold surface or MS encapsulated in MS and tethered onto CaP-collagen composite scaffolds (4 mm thick, 8 mm diameter). SDF-1α eluted from scaffolds into PBS will be collected at 2 day intervals over a 3 week period and placed in the lower chamber of the migration assay to observe differences in release kinetics and cell migration response. Migration studies will be carried out for 3, 6 and 12 hours. Post-experiment, by measuring the ratio of migratory cells in response to SDF-1α to cells migrating in the presence of control media (via GFP labeling), the induced migration response will be calculated as the ratio of chemotaxis induced migration to random migration.

Mechanical Bioreactor—Here, a multi-specimen 5200 BioDynamic Electroforce bioreactor (Test Instruments, MN) will be used to investigate the effect of cyclic mechanical loading and stem cell seeding on the changes in the mechanical properties of the graft and the cellular activity (proliferation, differentiation and signaling) within the graft. MSCs will be seeded directly on the CaP scaffold surface, while the HSCs will be seeded within a 5 mg/ml fibrin hydrogel (prepared from thrombin and fibrinogen) and cast within the CaP scaffold. The scaffolds will be exposed to cyclic mechanical conditioning for 2 hours/day, 1000 μstrains, at 1 Hz frequency. Static cell culture conditions will be used as controls. The media used will be cell growth media consisting of Dulbecco's Modified Eagle Medium, 10% fetal bovine serum and 100 U/ml each of penicillin and streptomycin. The 24 groups (8 samples/group/time point; at time points of 2, 4 and 6 weeks) evaluated will be all combinations of 3 groups with 2 SDF-1α dosages, 2 groups of biophysical conditions (static control, cyclic loading), and 4 groups of cell seeding conditions (no cells, MSCs on CaP only, HSCs in fibrin within CaP only, MSCs on CaP+HSCs in fibrin within CaP): 3×2×4=24 groups. Media will be collected on a regular basis and analyzed for soluble growth factors and extracellular Ca2+. Elastic modulus of the samples and media pH, O2 and CO2 concentration will be independently monitored for all samples throughout culture. At each time point, four samples will be randomly selected for plastic embedding and histological evaluation. The remaining 4 samples will be mechanically tested to failure as described in section 1.2 and resuspended in TRIzol (Invitrogen). Total RNA will be isolated using the RNeasy kit. Factors (Yap, Taz, connexin 43, Mmp13, Runx2, ERK1/2, FAK, RANKL, β-catenin, Akt1, Col1a1, FGF23, bmp2, hif1α and Vegf) involved in mechanotransduction and osteogenic-angiogenic coupling will be measured by q-PCR.

In Vivo Efficacy-Bone regeneration will be evaluated in the mandibular body of adult, skeletally mature Sprague Dawley rats. An SDF-1α group exhibiting the best bone regeneration outcome from the above study will be selected for this pilot study. Bone gaps will be created using sterile techniques under general anesthesia induced by an intraperitoneal injection of pentobarbital sodium (30 mg/kg) and will be geometrically similar to a rectangular full thickness defect (2.5×0.7×2 mm) with a final gap volume of 3.5 mm3.⁷³ All controls (traditional HA scaffolds) and treated groups (compliant-zone scaffolds with/without SDF-1α) will be secured using titanium fixation plates and Kirschner wires. Bone regeneration and function will be assessed at 2, 4, and 6 weeks after implantation. With 8 animals/group, 3 time points, and 3 groups, a total of 72 rats will be used. Outcome assessments will be performed using the μCT, histomorphometry and nano-indentation techniques on all samples (all this data is specifically noted in the management plan). Power analyses from a previous animal study⁷⁴ indicate that 8 animals/group will achieve a power of 0.9 with a=0.05. Sex will be treated as a biological variable (4 male and 4 female per group/time) and reported out for statistical impacts of sex in addition to treatment on time course. Histology will be performed by plastic embedding and bone regeneration will be assessed by staining with Sanderson's Rapid Bone Stain counterstained with van Gieson's picrofuchsin. Immunohistochemistry will be used to test for cell markers of mechano-transduction and the connexin hemi-channels to validate causality. Nano-indentation will be used to measure the local mechanical properties of both the residual scaffold as well as the newly formed callus and regenerated bone tissue at the different time points to evaluate regenerative efficacy.

### Example 2

Bioink formulations incorporating gelatin were systematically optimized to achieve reliable multi-material 3D printing of contiguous slow- and fast-dissolving calcium phosphate (CaP) segments while enabling the desired differential swelling and degradation behavior essential for creating sacrificial crumple zones. Gelatins of varying molecular weights, Bloom strengths (a measure of gel strength correlated with molecular weight and crosslinking potential), and processing methods (Type A acid-processed versus Type B alkaline-processed) were evaluated. Higher Bloom strength generally increases crosslink density and reduces mesh size within the hydrogel network, while Type A and Type B gelatins provide access to distinct isoelectric points (pI). Matching the bioink pH as closely as possible to the gelatin's pI minimizes electrostatic repulsion between chains, thereby enhancing physical crosslinking and modulating swelling and degradation kinetics.

Initial hydrogel samples (cut in half and submerged in 1 mL deionized water in 48-well plates) were monitored for swelling, mechanical consistency (rubbery vs. brittle behavior upon probing with a pipette tip), and degradation (residue formation or disintegration). Samples containing higher gelatin content or higher Bloom strength exhibited increased crosslink density, which initially slowed water uptake but sometimes led to excessive stiffness that compromised extrusion printability on the Bioplotter. In contrast, lower Bloom Type A gelatin improved flow characteristics without causing delamination between fast- and slow-dissolving CaP zones.

Formulations were tested in batches, including 80/20 and 90/10 HA/gelatin (w/w) ratios using blends of high- and low-Bloom Type A gelatin. Swelling and degradation were assessed over time (observations at approximately 1, 2, 3, and 4 hours post-immersion). Both formulations showed similar behavior initially, with slight dimensional changes and maintenance of a stiff, rubbery consistency. However, to further reduce excessive water uptake while preserving printability and the rapid swelling/degradation differential needed for sacrificial zones, formulations with lower overall gelatin concentration were prioritized. Blends of different Bloom strengths were also evaluated to fine-tune the hydrogel network, simulating the effects of antiplasticizers on mesh size and compliance.

Physical crosslinking strategies were explored to stabilize the gelatin phase without compromising the air-drying process or differential dissolution rates between β-TCP-rich (fast-dissolving) and HA-rich (slow-dissolving) segments. Dehydrothermal treatment was avoided due to excessive temperatures that could densify the CaP grains and diminish the crumple-zone effect. UV irradiation at 254 nm (multiple lower-dose exposures, with some samples receiving an additional 3-hour exposure) was tested as a surface crosslinking method. However, no significant differences in swelling, mechanical behavior, or degradation were observed between UV-treated and non-irradiated samples, indicating that physical crosslinking via pH-matched electrostatic interactions and optimized gelatin concentration was sufficient for the green-body scaffolds.

Subsequent iterations focused on the 65/35 HA/gelatin formulation (using blends of high- and low-Bloom Type A gelatin at reduced overall gelatin concentration). This ratio demonstrated markedly improved swelling and degradation profiles compared with higher-gelatin formulations: hydrogels swelled only modestly (~2-5% dimensional change) while retaining a stiff, rubbery consistency upon probing, even after 2-4 hours, with minimal softening or residue formation. Higher gelatin content or Bloom strength increased crosslink density and slowed degradation, whereas lower-Bloom Type A gelatin enhanced extrusion without excessive stiffness. The reduced-heat preparation protocol (evaporating most water prior to addition of HA and carboxymethylcellulose, while maintaining lower temperatures during gelatin and PEG200 incorporation) minimized thermal degradation of the gelatin and preserved printability. This approach also avoided physical aging effects observed upon rapid quenching below the glass transition temperature.

The optimized 65/35 formulations enabled reliable multi-head Bioplotter extrusion of layered fast- and slow-dissolving CaP zones with structural continuity and no delamination after air-drying. The preserved gelatin phase contributed to initial compliance and controlled Ca²⁺/PO₄³⁻ ion release, supporting early mechano-transductive signaling without compromising long-term scaffold integrity. Working formulations (e.g., 80/20 and 90/10 HA/gelatin with high/low Bloom blends) confirmed that reducing gelatin concentration, matching pH to the gelatin pI, and avoiding prolonged high-temperature exposure or sintering were critical to maintaining the rapid swelling and degradation differential in sacrificial segments while ensuring printability. These parameters were iteratively refined through non-working prototypes (e.g., high-Bloom gelatin causing poor extrudability or excessive stiffness, or sintered versions losing crumple efficiency) to define robust boundaries for the invention.

This optimized gelatin-stabilized ink system thus supports the fabrication of patient-specific, multi-material CaP scaffolds with spatially controlled sacrificial zones that progressively introduce localized compliant regions upon rehydration, activating YAP/TAZ-mediated pathways to re-initiate stalled bone regeneration in critical-size defects.

### REFERENCES

1. Erol B et al., Journal of Cranio-Maxillofacial Surgery. 2004; 32 (5): 308-13.
2. Gassner R et al., Journal of Cranio-Maxillofacial Surgery. 2003; 31 (1): 51-61.
3. Schuknecht B et al., Eur Radiol. 2005; 15 (3): 560-8.
4. Cochran D L. Journal of periodontology. 2008; 79 (8S): 1569-76.
5. Grossi S et al, Journal of periodontology. 1995; 66 (1): 23-9.
6. Moreau J L et al., Journal of Oral and Maxillofacial Surgery. 2007; 65 (12): 2503-11.
7. Perlyn C A et al., Plastic and reconstructive surgery. 2005; 115 (4): 1129-41.
8. Chana J S et al., Plastic and Reconstructive Surgery. 2004; 113 (1): 80-7.
9. Troulis M J et al., Journal of oral and maxillofacial surgery. 2004; 62 (3): 335-43.
10. Harashina T et al., Plastic and reconstructive surgery. 1978; 62 (4): 514-22.
11. Horiuchi K et al., Microsurgery. 1995; 16 (7): 450-4.
12. Perrott D H et al., International Journal of Oral and Maxillofacial Surgery. 1992; 21 (5): 260-5.
13. Keith et al., International Journal of Periodontics and Restorative Dentistry. 2006; 26 (4): 321.
14. Bradley J P et al., Plastic and reconstructive surgery. 2006; 118 (7): 1585-97.
15. Elsalanty M E et al., Journal of Craniofacial Surgery. 2007; 18 (6): 1397-402.
16. Kewitt G F et al., Journal of oral and maxillofacial surgery. 1999; 57 (12): 1419-25.
17. Makarov M R et al., Journal of oral and maxillofacial surgery. 1998; 56 (12): 1417-23.
18. Sawaki Y et al, Journal of Cranio-Maxillofacial Surgery. 1997; 25 (6): 310-5.
19. Van Strijen et al., Oral Surgery, Oral Medicine, Oral Pathology, Oral Radiology, and Endodontology. 2003; 96 (4): 392-7.
20. Boerckel J D et al., Biomaterials. 2011; 32 (22): 5241-51.
21. Kolambkar Y M et al., Bone. 2011. Epub 2011 May 31.
22. Guda T et al., Journal of Materials Science: Materials in Medicine. 2011; 22 (3): 647-56.
23. Stubbs D et al., Biomaterials. 2004; 25 (20): 5037-44.
24. Chesnutt B M et al., Journal of biomedical materials research Part A. 2009; 88 (2): 491-502.
25. Dias G J et al., Journal of biomedical materials research Part A. 2010; 95 (4): 1084-95.
26. Schek R M et al., Biomaterials. 2006; 27 (7): 1160-6.
27. Wahl D A, Czernuszka J T. Eur Cell Mater. 2006; 11:43-56.
28. Garrison K R et al., Cochrane Database Syst Rev. 2010 (6): CD006950.
29. Axelrad T et al., Journal of Bone & Joint Surgery, British Volume. 2008; 90 (12): 1617-22.
30. Nauth A et al., Journal of orthopaedic trauma. 2010; 24 (9): 543-6.
31. Perri B et al., The Spine Journal. 2007; 7 (2): 235-9.
32. Zara J N et al., Tissue Engineering Part A. 2011; 17 (9-10): 1389-99.
33. Guda T et al., The International journal of oral & maxillofacial implants. 2012; 27 (2): 288-94.
34. Guda T et al., Journal of biomaterials applications. 2014; 28 (7): 1016-27.
35. Guda T et al., Tissue engineering Part A. 2013; 19 (17-18): 1879-88.
36. Keogh M B et al., Acta Biomaterialia. 2010; 6 (11): 4305-13.
37. LeGeros R Z, Clinical Materials. 1993; 14 (1): 65-88.
38. Lieberman J R et al., JBJS. 2002; 84 (6): 1032-44.
39. Fong E L S et al., Biomaterials. 2011; 32 (2): 395-409.
40. Jung Y et al., Bone. 2006; 38 (4): 497-508.
41. Otsuru S et al., Stem cells (Dayton, Ohio). 2008; 26 (1): 223-34.
42. Zhu W et al., Journal of Biological Chemistry. 2011; 286 (30): 26794-805.
43. Guda T et al., Wound Repair and Regeneration. 2014; 22:39-49.
44. Marsell R, Einhorn T A. Injury. 2011; 42 (6): 551-5.
45. Kanczler J, Oreffo R. Eur Cell Mater. 2008; 15 (2): 100-14.
46. Azad T et al., Cells. 2019; 8 (5): 407.
47. Barreto S et al., Acta biomaterialia. 2017; 53:59-69.
48. Heng B C et al., Frontiers in Cell and Developmental Biology. 2020; 8:735.
49. Kegelman C D et al., Journal of Bone and Mineral Research. 2020; 35 (1): 196-210.
50. Chiou G et al., Cellular and molecular bioengineering. 2020; 13 (5): 507-26.
51. Morgan et al., The Journal of bone and joint surgery American volume. 2008; 90 (Suppl 1): 25.
52. Fang T D et al., Journal of bone and mineral research. 2005; 20 (7): 1114-24.
53. Tong L et al., Plastic and reconstructive surgery. 2003; 111 (1): 211-22.
54. Glatt V et al., The Journal of bone and joint surgery American volume. 2016; 98 (8): 677.
55. Glatt et al., JAAOS-Jour. American Academy of Orthopaedic Surgeons. 2016; 24 (7): e60-el.
56. Schmidt E C et al., OTA International. 2022; 5 (1 Suppl).
57. Boerckel et al., PNAS. 2011; 108 (37): E674-E80.
58. Liu C et al., Bone. 2018; 108:145-55.
59. McDermott A M et al., Science translational medicine. 2019; 11 (495).
60. Boerckel J D et al., Journal of Orthopaedic Research. 2012; 30 (7): 1067-75.
61. Herberg S et al., Science advances. 2019; 5 (8): eaax2476.
62. Glatt V et al., JBJS. 2021; 103 (3): 257-63.
63. Oest M E et al., Journal of Orthopaedic Research. 2007; 25 (7): 941-50.
64. Ong J L et al., Tissue Engineering Part C: Methods. 2022; 28 (7): 363-74.
65. Montelongo S A et al., Journal of Materials Science: Materials in Medicine. 2021; 32 (8): 1-16.
66. Meijer G J et al., Biomaterials. 2008; 29 (21): 3053-61.
67. Schmitz J P, Hollinger J O. Clinical Orthopaedics and Related Research. 1986; 205:299-308.
68. Koerten H, Van der Meulen J. Journal of biomedical materials research. 1999; 44 (1): 78-86.
69. Son J S et al., J Control Release. 2011.
70. Mirshahi F et al., Thrombosis Research. 2000; 99 (6): 587-94.
71. Young S et al., Tissue Engineering Part A. 2009; 15 (9): 2347-62.
72. Wernike E et al., Eur Cell Mater. 2010; 19:30-40.
73. Issa J P M et al., Clinical Oral Implants Research. 2009; 20 (11): 1286-92.
74. Guda T et al., Tissue Engineering Part C: Methods. 2014; 20 (9): 749-60.
75. Acosta F M et al., Tissue Engineering Part A. 2020; 26 (15-16): 905-14.
76. Pilia M et al., European Cells & Materials. 2014; 28:11-23.

1. A bone scaffold comprising at least a first segment comprising a slow-dissolving calcium phosphate (CaP) scaffold and at least a second sacrificial segment comprising a fast dissolving CaP scaffold, wherein the at least second sacrificial segment degrades and/or swells when exposed to aqueous fluid environments over a defined time course.

2. The bone scaffold of claim 1, further comprising one more segments comprised of calcium phosphate ceramics, other ceramics, or metals, wherein the additional segments have interconnected porosity, and wherein the bone scaffold has structural continuity between all segments.

3. The bone scaffold of claim 1, wherein the at least first segment and the at least second sacrificial segment are porous and have interconnected porosity.

4. The bone scaffold of claim 1, wherein the at least first segment and the at least second sacrificial segment have structural continuity throughout the scaffold.

5. The bone scaffold of claim 1, wherein the at least first segment and the at least second sacrificial segment have an interconnected porosity.

6. The bone scaffold of claim 1, wherein the scaffold is produced by 3D printing.

7. The bone scaffold of claim 1, wherein the at least second sacrificial segment contains at least one therapeutic agent.

8. The bone scaffold of claim 7, wherein the at least one therapeutic agent is a drug, a polymer, or a polypeptide.

9. The bone scaffold of claim 7, wherein the at least one therapeutic agent is stromal-derived factor-1 alpha (SDF-1α) encapsulated in poly-lactic-co-glycolic acid (PLGA) microspheres with a poly-ethyleneimine (PEI) coating, immobilized on surfaces of the scaffold and configured for controlled release to recruit mesenchymal stem cells without requiring supraphysiologic doses of bone morphogenetic proteins.

10. The bone scaffold of claim 1, wherein the at least second sacrificial segment is configured as repeating discrete layers having a thickness of 0.25 mm to 0.5 mm and spaced 1 mm to 2 mm apart along a longitudinal axis of the scaffold, such that degradation of the sacrificial segments progressively introduces compliant regions that reduce local stiffness and trigger YAP/TAZ-mediated mechano-transduction to re-initiate osteogenesis in non-healing central regions of the defect.

11. The bone scaffold of claim 1, further comprising a collagen wrap that at least partially surrounds the scaffold, wherein the collagen wrap mimics periosteum to guide intramembranous bone formation and enhance uniform bone bridging across the defect.

12. The bone scaffold of claim 1, wherein the scaffold is surrounded completely or partially by a wrap that is biocompatible and configured to support tissue integration.

13. The bone scaffold of claim 12, wherein the wrap is a collagen wrap.

14. A method of producing a segmented bone scaffold comprising:
printing at least a first segment using a first ink material having a first fluid dissolution rate when cured, and
printing at least a second sacrificial segment using a second ink material having a second fluid dissolution rate when cured,
wherein the bone scaffold is contiguous having structural continuity between the at least first and at least second sacrificial segments.

15. The method of claim 14, wherein the printing is on top of a metal stabilizing unit.

16. The method of claim 14, further comprising wrapping the segmented bone scaffold.

17. The method of claim 14, wherein printing is continuous, or discontinuous followed by drying of independently printed segments.

18. A method of treating a critical-size bone defect in a subject, comprising:
implanting the bone scaffold of claim 1 into the defect;
wherein degradation of the at least second sacrificial segment over time creates localized compliant regions that reduce mechanical stiffness, thereby activating mechano-transductive signaling in infiltrating cells to re-initiate stalled bone regeneration and promote continuous bridging of the defect without additional surgical intervention.

19. The method of claim 18, wherein the defect is a craniofacial or mandibular defect, and the scaffold is patient-specific, fabricated by 3D printing based on imaging data of the subject's anatomy.
